(19) United States
(12) Patent Application Publication
(10) Pub. No.: US 2025/0103953 A1
(43) Pub. Date: Mar. 27, 2025

(54) INFORMATION PROVIDING DEVICE, INFORMATION PROVIDING METHOD, AND RECORDING MEDIUM

(71) Applicant: NEC Corporation, Minato-ku, Tokyo (JP)

(72) Inventor: Yuki KOSAKA, Tokyo (JP)

(73) Assignee: NEC Corporation, Minato-ku, Tokyo (JP)

(21) Appl. No.: 18/565,608

(22) PCT Filed: Aug. 30, 2023
(86) PCT No.: PCT/JP2023/031529

§ 371 Date: Nov. 30, 2023

(30) Foreign Application Priority Data
Nov. 18, 2022 (WO) PCT/JP2022/042805

(51) Int. Cl. G06N 20/00

(52) U.S. Cl. CPC G06N 20/00

(57) ABSTRACT

An information providing device includes a physical information acquisition means that acquires physical information including an attribute, a physical condition, and a goal with respect to the physical condition of a user, a user information acquisition means that acquires information regarding the user including schedule information of the user, a decision means that decides a content of an advice for the goal and a timing of executing the content of the advice based on the physical information and the schedule information, and an output means that outputs the content of the advice and the timing.

### TECHNICAL FIELD

The present disclosure relates to an information providing device, an information providing method, and a recording medium.

### BACKGROUND ART

In order to encourage the user to improve his or her lifestyle, there is a technique in which the user is advised on daily behavior based on a behavior log of the user.

For example, PTL 1 discloses a health management server that generates an appropriate advice message from meals or a health condition of a health management subject. Furthermore, PTL 2 discloses an information processing device that transmits an advice for bringing a physical condition closer to an ideal physical body specified by the user based on lifestyle information and preference information of the user.

### CITATION LIST

### Patent Literature

PTL 1: WO 2017/022013 A1

PTL 2: WO 2019/116679 A1

### SUMMARY OF INVENTION

### Technical Problem

However, with the techniques described in PTL 1 and PTL 2, the user is not sometimes able to behave according to the advice due to the user's schedule. Therefore, it is necessary to give an advice that can be executed by the user.

An example of an object of the present disclosure is to provide an information providing device capable of providing an advice for lifestyle improvement that can be executed by a user.

### Solution to Problem

An information providing device according to an aspect of the present disclosure includes a physical information acquisition means that acquires physical information including an attribute, a physical condition, and a goal with respect to the physical condition of a user, a user information acquisition means that acquires information regarding the user including schedule information of the user, a decision means that decides a content of an advice for the goal and a timing of executing the content of the advice based on the physical information and the schedule information, and an output means that outputs the content of the advice and the timing.

An information providing method performed by a computer according to an aspect of the present disclosure includes acquiring physical information including an attribute, a physical condition, and a goal with respect to the physical condition of a user, acquiring information regarding the user including schedule information of the user, deciding a content of an advice for the goal and a timing of executing the content of the advice based on the physical information and the schedule information, and outputting the content of the advice and the timing.

A recording medium according to an aspect of the present disclosure stores a program for causing a computer to execute acquiring physical information including an attribute, a physical condition, and a goal with respect to the physical condition of a user, acquiring information regarding the user including schedule information of the user, deciding a content of an advice for the goal and a timing of executing the content of the advice based on the physical information and the schedule information, and outputting the content of the advice and the timing. Advantageous Effects of Invention

According to the present disclosure, it is possible to provide an advice for lifestyle improvement that can be executed by a user.

### BRIEF DESCRIPTION OF DRAWINGS

FIG. 1 is a block diagram illustrating an example of a configuration of an information providing device according to a first example embodiment.

FIG. 2 is a diagram illustrating a hardware configuration in which the information providing device according to the first example embodiment is implemented by a computer device and its peripheral device.

FIG. 3 is a graph illustrating a frequency at which a user has stayed at a place in the first example embodiment.

FIG. 4 is an example of a screen for a physical information acquisition unit to acquire physical information in the first example embodiment.

FIG. 5 is an example of a screen for the user to choose a content of an advice in the first example embodiment.

FIG. 6 is an example in which schedule information of the user is output in the first example embodiment.

FIG. 7 is a diagram for explaining a method of deciding an advice in the first example embodiment.

FIG. 8 is a flowchart illustrating an operation of the information providing device in the first example embodiment and a modification of the first example embodiment.

FIG. 9 is a diagram for explaining a method of deciding a content of an advice based on behavior information and lifestyle information in another modification of the first example embodiment.

FIG. 10 is a diagram for explaining a method of deciding a content of an advice based on behavior information and preference information in another modification of the first example embodiment.

FIG. 11 is a diagram for explaining a method of deciding a content of an advice based on environment information in another modification of the first example embodiment.

FIG. 12 is a block diagram illustrating an example of a configuration of an information providing device according to a second example embodiment.

FIG. 13 is a block diagram illustrating an example of a configuration of an information providing device according to a third example embodiment.

FIG. 14 is a flowchart illustrating an operation of the information providing device in the third example embodiment.

### EXAMPLE EMBODIMENT

Hereinafter, example embodiments of an information providing device, an information providing method, and a non-transitory recording medium storing a program according to the present disclosure will be described in detail with reference to the drawings. These example embodiments do not limit the technology according to the disclosure.

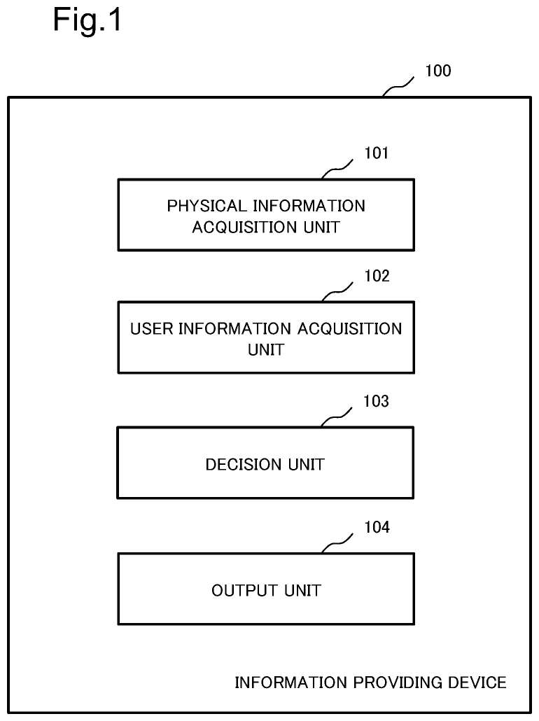

FIG. 1 is a block diagram illustrating an example of a configuration of an information providing system according to a first example embodiment. Referring to FIG. 1, the information providing device 100 includes a physical information acquisition unit 101, a user information acquisition unit 102, a decision unit 103, and an output unit 104.

The information providing device 100 outputs an advice on a behavior change for lifestyle improvement to a user. For example, the information providing device 100 has physical information registered on an application program for health management, the physical information including an attribute, a physical condition, and a goal with respect to the physical condition of the user, and outputs an advice to the user on the same application program. In the present example embodiment, the advice is an advice on a behavior to be performed by the user to improve an index indicating a health condition such as a weight and a height, an abdominal circumference, a blood pressure, a blood glucose level, and a blood lipid of the user. The behaviors include contents of exercise and meal. In this manner, for example, the information providing device 100 promotes the user to perform his/her own healthcare (self-care).

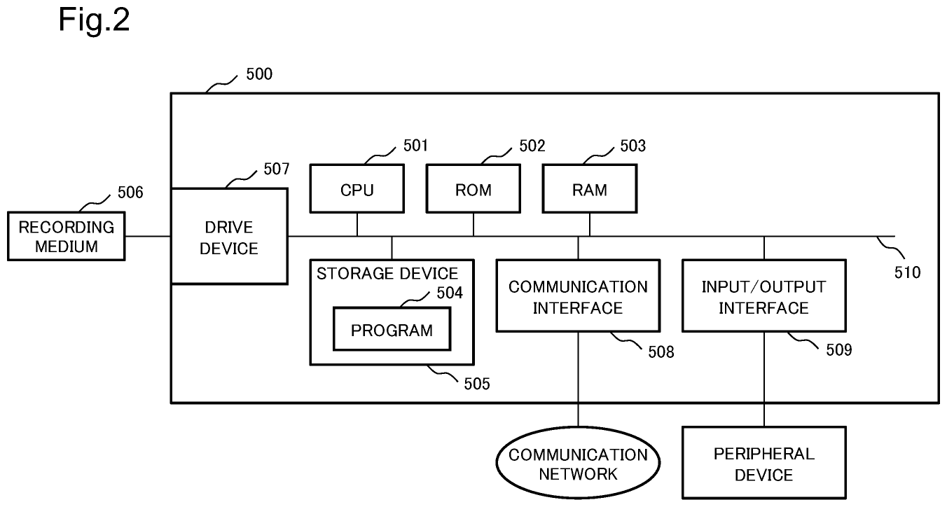

FIG. 2 is a diagram illustrating an example of a hardware configuration in which the information providing device 100 according to the first example embodiment of the present disclosure is implemented by a computer device 500 including a processor. As illustrated in FIG. 2, the information providing device 100 includes a central processing unit (CPU) 501, memories such as a read only memory (ROM) 502 and a random access memory (RAM) 503, a storage device 505 such as a hard disk that stores a program 504, a communication interface 508 for network connection, and an input/output interface 509 that inputs and outputs data. In the first example embodiment, the information providing device 100 is connected to each component via a bus 510. In addition, the information providing device 100 according to the first example embodiment illustrated in FIG. 1 may be configured by cloud computing or the like.

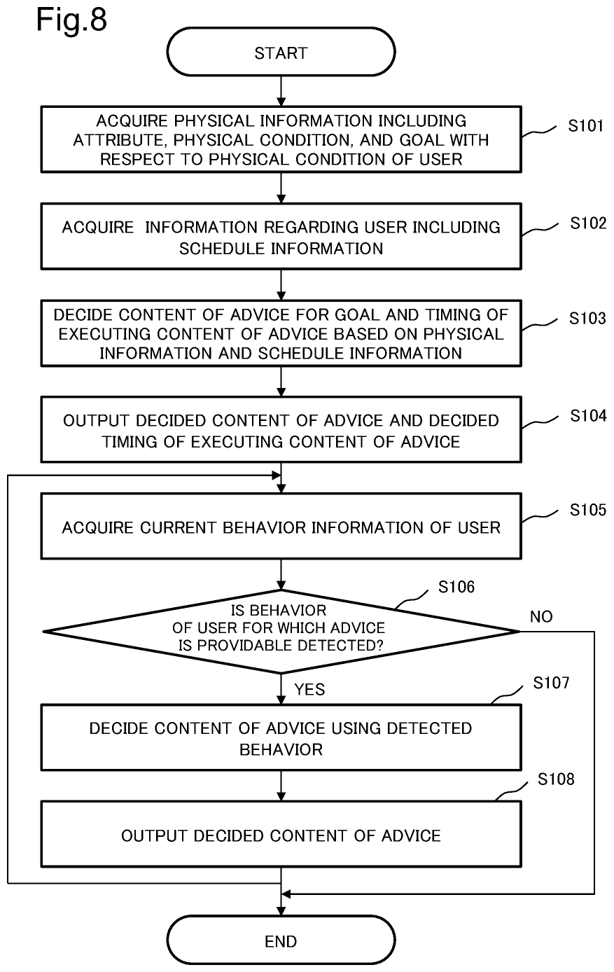

The CPU 501 operates an operating system to control the entire information providing device 100 according to the first example embodiment of the present invention. In addition, the CPU 501 reads a program or data from a recording medium 506 mounted on, for example, a drive device 507 to a memory. In addition, the CPU 501 functions as the physical information acquisition unit 101, the user information acquisition unit 102, the decision unit 103, the output unit 104, or some of them in the first example embodiment, and executes a process or a command in a flowchart illustrated in FIG. 8 to be described later based on the program.

The recording medium 506 is, for example, an optical disk, a flexible disk, a magneto-optical disk, an external hard disk, a semiconductor memory, or the like. The semiconductor memory or the like, which is a part of the recording medium, is a non-volatile storage device, records the program therein. Alternatively, the program may be downloaded from an external computer connected to a communication network although not illustrated.

As described above, the first example embodiment illustrated in FIG. 1 is implemented by the computer hardware illustrated in FIG. 2. However, the means for implementing each of the units included in the information providing device 100 of FIG. 1 is not limited to the configuration described above. Furthermore, the information providing device 100 may be implemented by one physically coupled device, or may be realized by a system including two or more physically separated devices connected in a wired manner or in a wireless manner.

The physical information acquisition unit 101 is a means for acquiring physical information including an attribute, a physical condition, and a goal with respect to the physical condition of the user. The attribute of the user includes a gender and an age. The physical condition is a most recent measurement value of a health index indicating a health condition such as a weight and a height, an abdominal circumference, a blood pressure, a blood glucose level, and a blood lipid. The goal is an ideal value with respect to the index, and includes a time limit until the target condition is achieved. The goal is, for example, reduction of the weight by 0.5 kg after 1 month. In the present example embodiment, the goal may be a final goal (e.g., a reduction by 5 kg after half a year) or a short-term goal (e.g., a weekly goal) that is subdivided to accomplish the final goal.

The physical information acquisition unit 101 acquires, for example, an attribute, a physical condition, and a goal with respect to the physical condition input to the application program. In addition, the physical information acquisition unit 101 may acquire the attribute from a terminal owned by the user as long as the attribute is registered in the terminal. The physical information acquisition unit 101 may acquire information indicating the physical condition from each health index measuring instrument through the communication interface 508 as long as each measuring instrument is connected to the network. In addition, if an information management server of a medical institution holding information indicating the physical information is connected to the network, the physical information acquisition unit 101 may acquire the information indicating the physical condition from the information management server through the communication interface 508.

The physical information acquisition unit 101 may input the attribute and the physical condition for a goal, and acquire the goal for achieving an ideal situation based on a learning model trained by machine learning. This learning model refers to a prediction model that is machine-trained for a plurality of users using, as learning data, an attribute of each user a pair of pieces of information including a physical condition of the user determined in advance before X months (e.g., 3, 6, or 12 months ago) and current physical information of the user, and predicts physical information of the user after X months when the attribute of the user and the current physical condition of the user are input.

The user information acquisition unit 102 is a means for acquiring information regarding the user including schedule information of the user. The schedule information includes user's schedules after a current point of time such as work/school, exercise, meal, and house (home), and particularly, schedules that affect a content of an advice for life improvement. The user information acquisition unit 102 acquires, for example, schedule information stored in the terminal of the user.

In addition, the user information acquisition unit 102 may acquire life log information of the user and estimate schedule information of the user based on the life log information. In the present example embodiment, the life log information is information regarding behaviors such as work, meal, exercise, and sleep, and includes a content of each behavior, place information, a required time, a movement time, a movement route, and the like.

In order to estimate schedule information of the user, the user information acquisition unit 102 may acquire information on places where the user was staying based on the life log information. The place information is information regarding places where the user is living in daily life, and examples thereof include work/school, exercise, meal, and home. In the present example embodiment, the “staying” refers to, for example, staying in the same range (e.g., less than 200 m) for a predetermined time (e.g., 20 minutes or more). The user information acquisition unit 102 specifies place information such as longitude and latitude from GPS location information of the terminal of the user, and specifies types of places based on the correspondence with map information. The user information acquisition unit 102 specifies a place where the user is staying from a place information type set in advance (work/school, exercise, meal, house). In a case where there is no place corresponding to the place information type set in advance, the user information acquisition unit 102 may inquire of the user about the place where the user is staying to input the place. Furthermore, the user information acquisition unit 102 may add the input information as a place type. Furthermore, the user information acquisition unit 102 may acquire a motion such as walking of the user based on information obtained from an acceleration sensor of the terminal of the user.

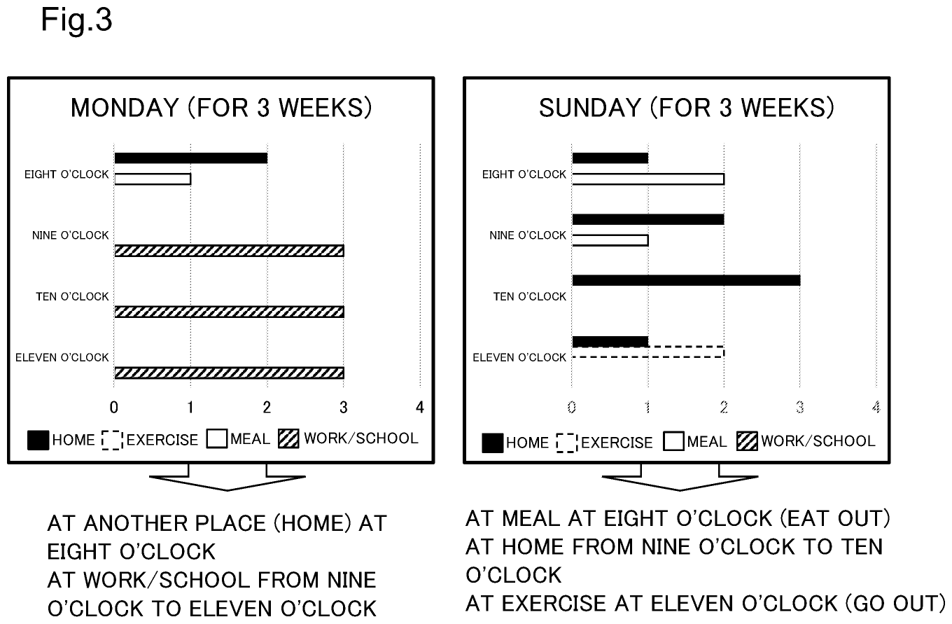

Furthermore, the user information acquisition unit 102 may acquire a frequency at which the user has stayed at a place. FIG. 3 is a graph illustrating a frequency at which the user has stayed at a place. In the example of FIG. 3, a frequency at where the user has stayed at a place in each time zone is illustrated for each day of the week. For example, the user information acquisition unit 102 estimates information on a place where the user was in each time zone for each day of the week based on the information on places where the user have been stayed and frequencies at which the user have been stayed at the places.

The decision unit 103 is a means for deciding a content of an advice for a goal and a timing of executing the content of the advice based on the physical information and the schedule information. Specifically, first, the decision unit 103 calculates calories to be reduced in order for the user to approach the target physical condition based on the physical information including the goal with respect to the physical condition of the user. Next, based on the schedule information of the user, the decision unit 103 extracts a timing such as a day or a time zone at which a behavior for reducing calories is performed. Next, the decision unit 103 decides to give an advice so that the behavior is performed at the extracted timing.

As the content of the advice decided by the decision unit 103, it may be simply advised to increase the amount of exercise or reduce the calorie intake of the meal, or a particular content of an exercise or a meal may be specifically advised. Furthermore, the decision unit 103 may receive an answer of the user in order to decide a content of an advice a timing of executing the advice. For example, the decision unit 103 may receive an answer as to whether to increase the amount of exercise and decrease the calorie intake of the meal. Furthermore, in order to reduce calories by doing an exercise, the decision unit 103 may receive the type of the exercise to be done by the user.

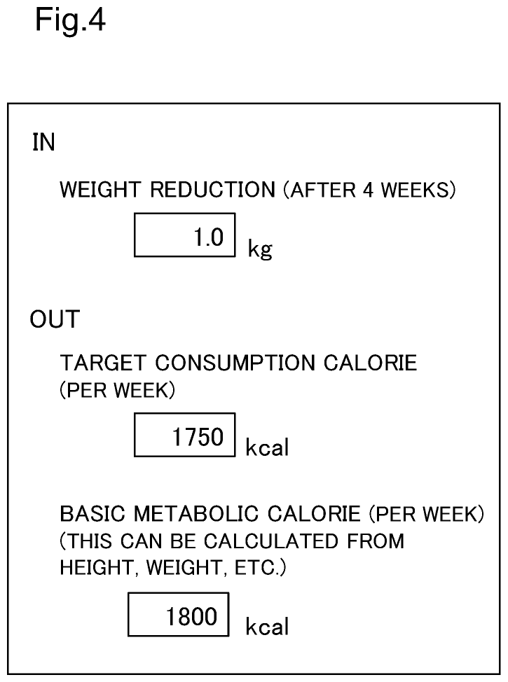

Here, a method of deciding a content of an advice to be provided to the user and a timing of executing the advice will be described with reference to the drawings. FIG. 4 is an example of a screen for the physical information acquisition unit 101 to acquire physical information. In the example of FIG. 4, a weight reduction by 1.0 kg after 4 weeks is input as a goal with respect to the physical condition. As illustrated in FIG. 4, the decision unit 103 may calculate information regarding target calorie consumption and basic metabolic calories, and the output unit 104 may output these pieces of information. In the example of FIG. 4, in order for the user to reduce 1.0 kg, it is necessary to reduce 7000/4 kcal=1750 kcal per week. That is, it is necessary to reduce 1750/7=250 kcal per day. Therefore, for example, the decision unit 103 advises three types of exercises each reducing 100 kcal every day.

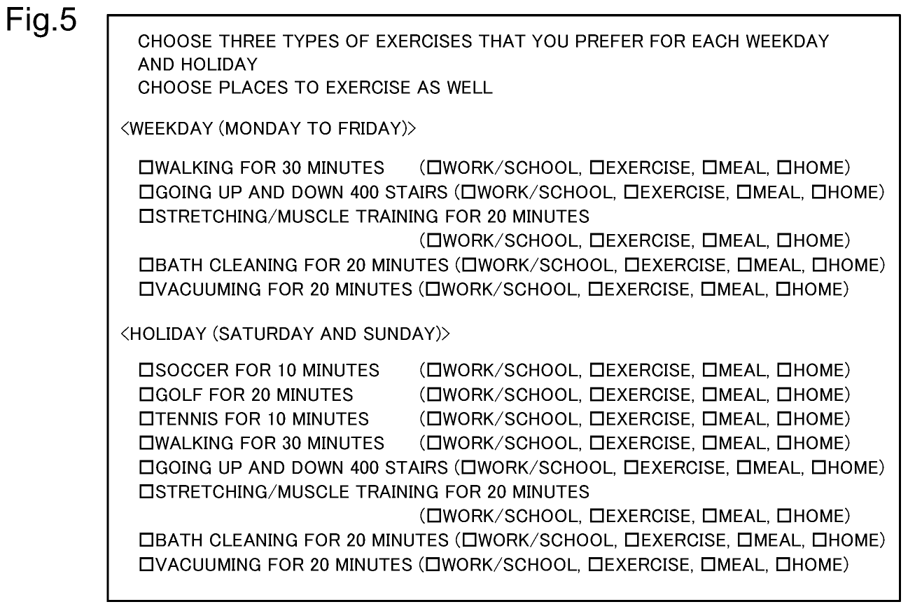

FIG. 5 is an example of a screen for the user to choose a content of an advice. In the example of FIG. 5, an advice on exercise is displayed to choose any of walking, going up and down stairs, stretching/muscle training, bath cleaning, and vacuuming for weekdays (Monday to Friday), and to choose sports such as soccer, golf, and tennis for holidays (Saturday and Sunday) in addition to the options for weekdays. Furthermore, in the example of the screen of FIG. 5, for each exercise, a check is input to a place (place information type) where the exercise can be executed among work/school, exercise, meal, and home.

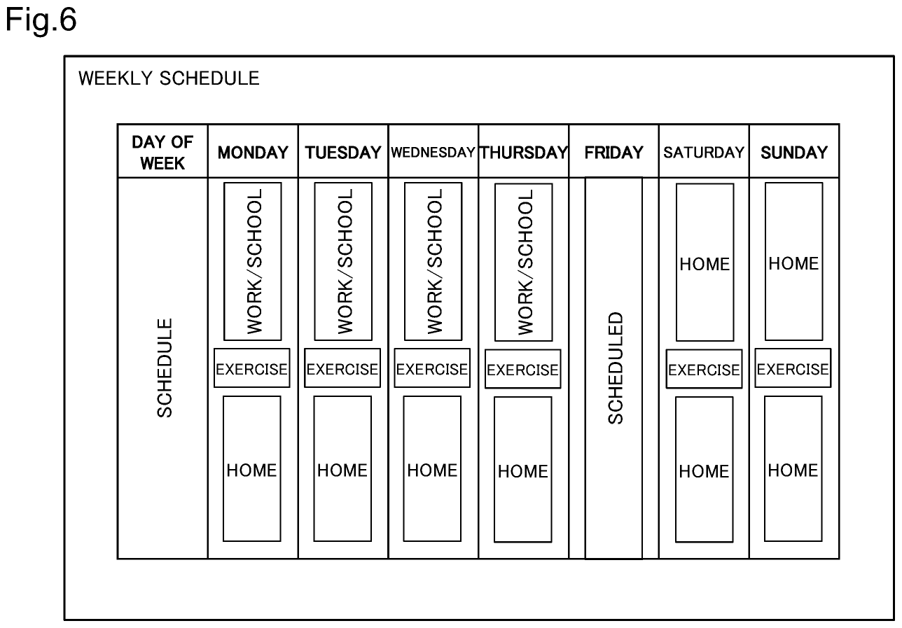

In the example of the screen of FIG. 5, when an input of an exercise type and a scheduled place at which the exercise can be executed is received from the user, the user information acquisition unit 102 acquires schedule information of the user. The user information acquisition unit 102 may acquire schedule information estimated based on the information on places where the user have been stayed and frequencies at which the user have been stayed at the places. FIG. 6 illustrates an example in which the schedule information of the user is output in the present example embodiment. Next, the decision unit 103 specifies time zones (timings) in which the user can carry out the exercises based on the exercises chosen by the user on the screen of FIG. 5 and the places where the exercises can be carried out, and the place information types such as work/school, exercise, meal, and home extracted from the schedule information. Next, the decision unit 103 extracts a day of the week for which no place information type is included from the schedule information of the user. In a case where one or more days of the week are extracted, the exercises for the extracted days of the week are allocated to days of the week other than the extracted days of the week. Specifically, in the example of FIG. 6, no place information type is included for Friday. In this case, the decision unit 103 decides to advise three types of exercises for Saturday and three types of exercises for Friday to be performed on Saturday, without giving an advice on three types of exercises to be performed on Friday.

For example, the decision unit 103 decides an exercise type to be advised and a timing to be advised as to a schedule time zone of a day of the week., from the acquired schedule information of the user and the information on the exercises and places (place information types: work/school, exercise, meal, home) chosen by the user in FIG. 5 where the exercises can be performed. In addition, it is assumed that the user is scheduled to stay at home for three consecutive hours in a daytime time zone. In this case, in the example of the screen of FIG. 5, if “home” is chosen as a scheduled place where an exercise can be performed, the decision unit 103 may decide to give an advice on a content of the exercise that can be performed at home in the time zone when the user stays at home.

The output unit 104 is a means for outputting the content of the advice and a timing of executing the content of the advice. The output unit 104 outputs, for example, the decided content of the advice and the decided timing of executing the content of the advice on the application program. Furthermore, the output unit 104 may send a message to the user based on the information decided by the decision unit 103 as to which exercise type is to be advised in which schedule time zone on which day of the week. Specifically, if the decision unit 103 decides an advice on an exercise for Wednesday in FIG. 6 is “walking for 30 minutes” and “work/school” as a time zone, the output unit 104 sends a message “it is recommended to walk for 30 minutes” to the user in the decided time zone. The output unit 104 may function as an output control means for a display device or a voice output device that is a user terminal owned by the user to perform an output. For example, the output unit 104 causes the display device to display the content of the advice and the timing of executing the content of the advice. In this case, the output unit 104 generates a display signal and supplies the generated display signal to the display device. The output unit 104 may perform a sound output control of the voice output device to output voice guidance or the like to be notified to the user.

As described above, in the information providing device 100 according to the first example embodiment, the decision unit 103 decides a content of an advice for a goal and a timing of executing the content of the advice based on the physical information and the schedule information, and the output unit 104 outputs the content of the advice and the timing of executing the content of the advice. In this case, for example, in a case where the user has a full day's schedule and it is not possible to secure time to execute a behavior for improving the lifestyle, it is possible to encourage the user to execute the content of the advice for the fully scheduled day on another day. This makes it possible to provide an advice for lifestyle improvement that can be executed by the user. For example, the user can behave to improve lifestyle with reference to the advice. That is, the information providing device 100 can support the decision making of the user by providing an advice for lifestyle improvement.

### Modification of First Example Embodiment

Next, a modification of the first example embodiment of the present disclosure will be described in detail with reference to the drawings. Hereinafter, description overlapping with what has been described above will be omitted unless the omission obscures the description of the present example embodiment.

In the first example embodiment described above, the decision unit 103 decides a content of an advice for a goal and a timing of executing the content of the advice, and the output unit 104 outputs these contents. On the other hand, in the present modification, the user information acquisition unit 102 further acquires current behavior information of the user. Then, when a behavior for which an advice is providable is detected, the decision unit 103 decides a content of the advice using the detected behavior. In the present modification, the advice decided by the decision unit 103 is an advice regarding a content of a specific exercise or meal that can be performed in the current behavior of the user.

The user information acquisition unit 102 acquires, as the current behavior information of the user, position information of the user based on, for example, a position of the terminal of the user using a global positioning system (GPS), a combination of Wi-Fi and GPS, and Bluetooth low energy (registered trademark). Furthermore, the user information acquisition unit 102 may acquire schedule information at a current time from the schedule information saved in the terminal of the user. However, the behavior information acquired by the user information acquisition unit 102 is not limited to these kinds of information as long as a current behavior of the user can be grasped. For example, when the user is moving, the user information acquisition unit 102 acquires current behavior information of the user and outputs the acquired information to the decision unit 103.

When detecting a behavior for which an advice is providable, the decision unit 103 decides a content of the advice using the detected behavior. For example, when detecting that the user is about to get on an elevator based on the position information of the user, the decision unit 103 decides to advise the user to use stairs. In addition, when the schedule information of the user at a current time is soccer and it is detected that the user has moved to a soccer field, the decision unit 103 decides to advise the user to play soccer for a predetermined time. In addition, when it is detected that the user has moved to a convenience store, the decision unit 103 decides to advise the user to purchase food having a small calorie intake. Furthermore, the output unit 104 outputs the decided content of the advice to the user at any time using the application program or a message.

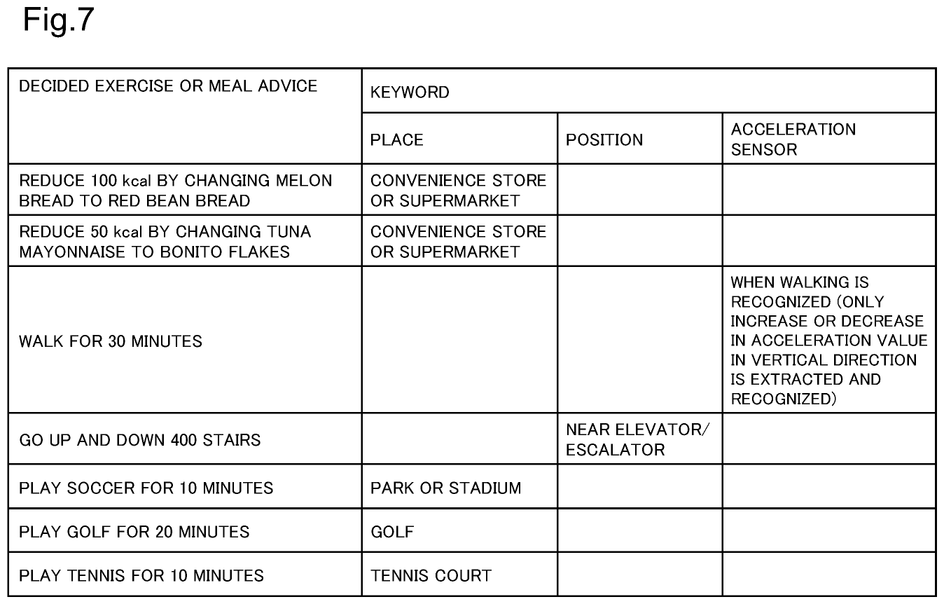

FIG. 7 is a diagram for explaining a method of deciding an advice by the decision unit 103. As illustrated in FIG. 7, keywords related to information on a place, a position, and an acceleration sensor are associated with each advice on an exercise or a meal in advance. The decision unit 103 decides whether a behavior corresponds to the keyword based on the information on the place, the position, and the acceleration sensor acquired from the terminal of the user. When the behavior corresponds to the keyword, the decision unit 103 decides the associated content of the advice. As a result, the decision unit 103 can timely give an advice without inquiring the user.

FIG. 8 is a flowchart illustrating an outline of an operation of the information providing device 100 in the first example embodiment and the modification of the first example embodiment. Note that the process according to this flowchart may be executed based on the program control by the processor described above. Note that, in this flowchart, the processing in S101 to S104 corresponds to a part of the first example embodiment, and the processing in S105 to S108 corresponds to a part of the modification of the first example embodiment.

As illustrated in FIG. 8, the physical information acquisition unit 101 acquires physical information including an attribute, a physical condition, and a goal with respect to the physical condition of the user (step S101). Next, the user information acquisition unit 102 acquires information regarding the user including schedule information (step S102). Next, the decision unit 103 decides a content of an advice for a goal and a timing of executing the content of the advice based on the physical information and the schedule information (step S103). The output unit 104 outputs the decided content of the advice and the decided timing of executing the content of the advice (step S104). Note that the processing in S101 to S104 is performed, for example, at a time interval for outputting a short-term goal (for example, on a weekly basis).

Next, the user information acquisition unit 102 acquires current behavior information of the user (step S105). When detecting a behavior of the user for which an advice is providable (step S106; YES), the decision unit 103 decides a content of an advice using the detected behavior (step S107). Next, the output unit 104 outputs the decided content of the advice (step S108). The processing in S105 to S108 is executed each time the decision unit 103 detects a behavior of the user for which an advice is providable.

On the other hand, when the decision unit 103 does not detect any behavior of the user for which an advice is providable within a predetermined period (for example, until the deadline of the short-term goal) (step S106; NO), the process ends. Accordingly, the information providing device 100 ends the information providing process.

As described above, in the information providing device 100 according to the modification of the first example embodiment, when detecting a behavior of the user for which an advice is providable, the decision unit 103 decides a content of an advice using the detected behavior. Next, the output unit 104 outputs the decided content of the advice. In this case, for example, the user can behave for life improvement in a timely manner in his/her normal life.

### Second Modification of First Example Embodiment

Next, another modification of the first example embodiment of the present disclosure will be described. Hereinafter, description overlapping with what has been described above will be omitted unless the omission obscures the description of the present example embodiment. In the present modification, in order to decide a content of an advice to the user, the decision unit 103 uses any one of life log information and environment information together with the current behavior information of the user, in addition to the schedule information of the user.

The user information acquisition unit 102 acquires schedule information, current behavior information, and life log information of the user. A method of acquiring each type of information is similar to that in the first example embodiment or the first modification of the first example embodiment. In the present modification, for example, the decision unit 103 decides a content of an advice based on the behavior information, the life log information, and lifestyle information and preference information of the user obtained by analyzing the life log information.

The lifestyle information is information regarding daily meal and exercise, and is information acquired by analyzing a behavior history the user such as a movement history, a movement time, and a commuting route, or information regarding meal such as a food and drink purchase history, a visit to a restaurant, a detail of an order in a restaurant, and an image captured during a meal. The user information acquisition unit 102 acquires, for example, information such as daily exercise content, amount of exercise, calorie consumption, or the like calculated based on the behavior history of the user as information regarding exercise. In addition, the user information acquisition unit 102 acquires, for example, information such as a meal content and calorie intake that the user takes daily, as information regarding meal. The user information acquisition unit 102 may estimate lifestyle information from the life log information input onto the application program.

Here, an example in which lifestyle information is estimated based on the life log information input onto the application program will be specifically described. As the lifestyle information, (1) favorite places and exercise types, and (2) favorite restaurants and menu names will be described as an example. (1) Regarding the favorite places and exercise types, for example, the user is requested to input a place where an exercise was performed and an exercise type. Based on the result, combinations of places and exercise types are ranked in descending order of the total number of times in two weeks, and the top three combinations of places and exercise types are set as the favorite places and exercise types. (2) Similarly, regarding favorite restaurants (eating plates), for example, when the user eats out at a restaurant, the user is requested to input a restaurant name and a menu name. Based on the result, combinations of restaurant names and menu names are ranked in descending order of the total number of times in two weeks, and for example, the top three combinations of restaurants and menu names are set as the favorite restaurants and menu names.

The preference information is, for example, information regarding user's preference with respect to items of personal preference such as tea, coffee, and confectionery, or information regarding user's hobbies such as favorite sports. The preference information is acquired by analyzing, for example, a camera mounted on the user terminal, a purchase history, and the like. In addition, the user information acquisition unit 102 may estimate preference information from the life log information input onto the application program. In this case, for example, the user is requested to directly input contents of foods that the user has eaten onto the application program. The results are aggregated, and for example, the rankings are created in descending order of the total number of times in two weeks. For example, the top three food are estimated as favorite foods.

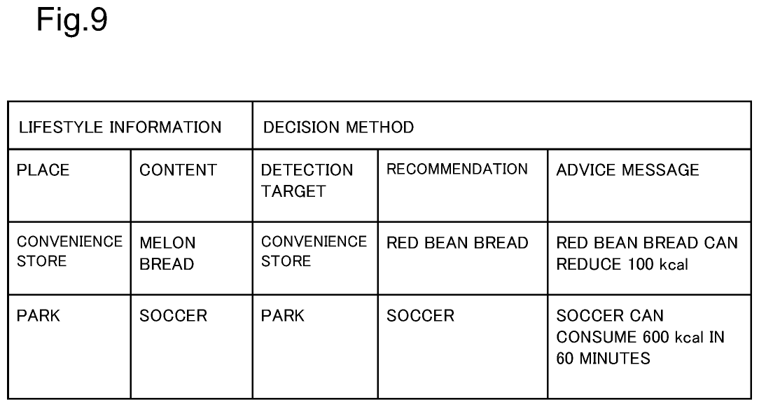

FIG. 9 is a diagram for explaining a method of deciding a content of an advice based on behavior information and lifestyle information in the present modification. As illustrated in FIG. 9, behavior information (place) detected as lifestyle information, a recommendation, and a content of an advice are stored in the storage device 505 in association with each other. The lifestyle information in the example of FIG. 9 indicates that the user has a habit of eating melon bread in a convenience store and a habit of playing soccer in a park. When detecting a specific behavior, the decision unit 103 may decide to give an advice associated with the behavior information. In the example of FIG. 9, when detecting that the user is in a convenience store, the decision unit 103 decides to give an advice to change from melon bread to red bean bread with a smaller calorie intake than the melon bread, and the output unit 104 sends a message “red bean bread can reduce 100 kcal”. Furthermore, when detecting that the user is in a park, the decision unit 103 decides to give an advice to play soccer, and the output unit 104 sends a message, for example, “soccer can consume 600 kcal in 60 minutes”.

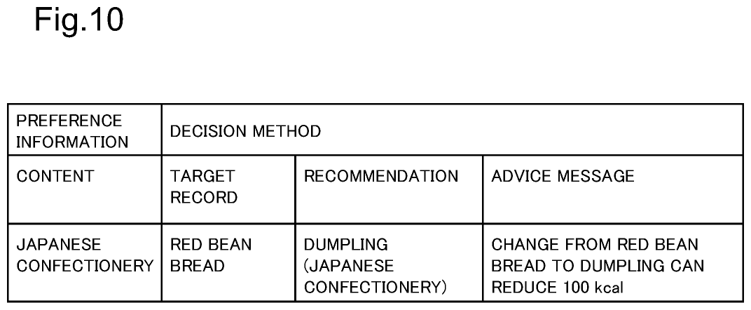

FIG. 10 is a diagram for explaining a method of deciding a content of an advice based on behavior information and preference information in the present modification. As illustrated in FIG. 10, the preference information and the advice decision method are stored in the storage device 505 in association with each other. In the example of FIG. 10, it is shown regarding Japanese confectionery that the user prefers red bean bread. When the user who likes red bean bread records that the user has eaten red bean bread on the application program, the decision unit 103 advises the user to change the red bean bread to a dumpling with a smaller calorie intake. The output unit 104 sends a message, for example, “the change from red bean bread into a dumpling can reduce 100 kcal”.

As another example, when detecting that the user has moved to a ramen noodle restaurant, the decision unit 103 may decide to advise the user to eat ramen noodle with a smaller calorie intake. Furthermore, it is assumed that the calorie intake of ramen noodle is higher in the order of salt→soy sauce→soybean paste. In this case, for example, if the user eats ramen noodle, the decision unit 103 may decide to give an advice “salt is recommended because the calorie intake is higher in the order of salt→soy sauce→soybean paste”. Furthermore, as another example, if the user who is to be advised to exercise on Saturday likes to play soccer, the decision unit 103 may decide to advise the user, for example, to play soccer for a predetermined time. In addition, for example, when detecting that the user who likes to play soccer in a park has moved to the park, the decision unit 103 may decide to advise the user to play soccer in the park for a predetermined time.

Furthermore, in the present modification, the user information acquisition unit 102 may further acquire environment information regarding a current environment around the user, and the decision unit 103 may decide a content of an advice based on the environment information. The environment information refers to, for example, weather, temperature, humidity, and the like around the position information of the user, and the user information acquisition unit 102 acquires the environment information based on weather forecast information and the like.

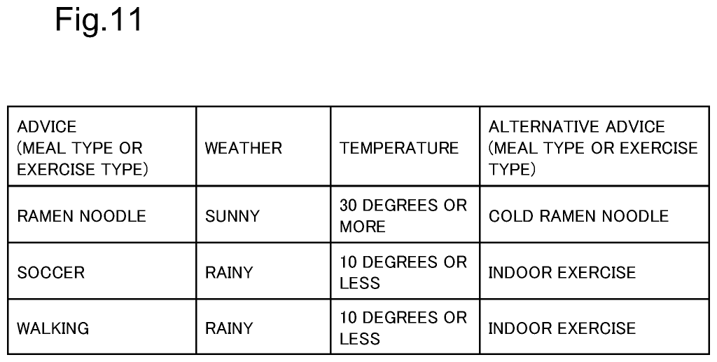

FIG. 11 is a diagram for explaining a method of deciding a content of an advice based on environment information by the decision unit 103. As illustrated in FIG. 11, the storage device 505 stores a content of an alternative advice for changing the content of the advice stored in advance in a specific weather or at a specific temperature in association with each other. In the example of FIG. 11, if the temperature is higher than a predetermined temperature (e.g., 30 degrees or more), the decision unit 103 gives an advice “cold ramen noodle is recommended, and salt is recommended because the calorie intake is higher in the order of salt→soy sauce→soybean past” to a user who often eats ramen noodle at a favorite restaurant or a user whose favorite food is ramen noodle, instead of “if you eat ramen noodle, salt is recommended because the calorie intake is higher in the order of salt→soy sauce→soybean paste”, which is normally advised. Furthermore, when the weather is rainy, the decision unit 103 does not advise a user who is scheduled to play soccer on the rainy day of the week or a user who likes soccer to play soccer. Alternatively, instead of advising the user to play soccer, indoor sports or indoor exercises are presented. In this case, it is possible to provide a content of an advice that is more likely to be executed by the user.

### Second Example Embodiment

Next, a second example embodiment of the present disclosure will be described in detail with reference to the drawings. Hereinafter, description overlapping with what has been described above will be omitted unless the omission obscures the description of the present example embodiment.

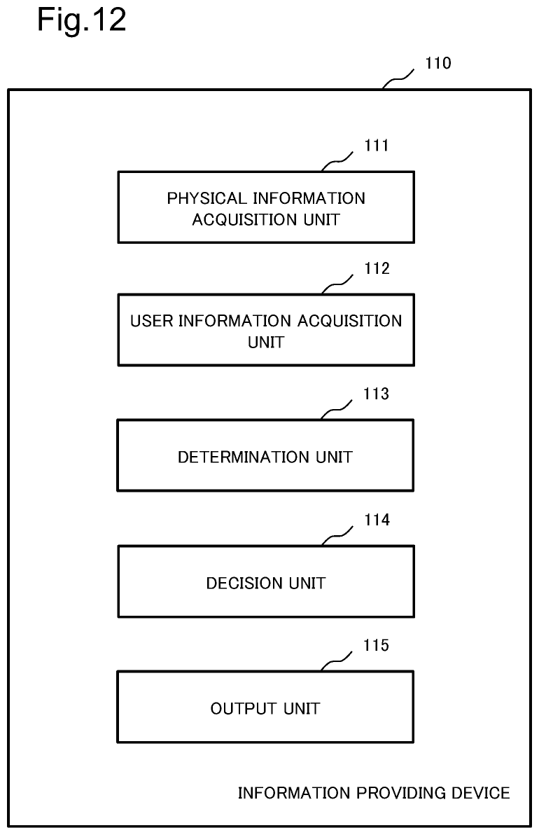

FIG. 12 is a block diagram illustrating an example of a configuration of an information providing device 110 according to the second example embodiment. The function of the information providing device 110 can be implemented not only by hardware similarly to the computer device illustrated in FIG. 2 but also by a computer device based on program control or by software.

As illustrated in FIG. 12, the information providing device 110 includes a physical information acquisition unit 111, a user information acquisition unit 112, a determination unit 113, a decision unit 114, and an output unit 115. The information providing device 110 is different from that according to the first example embodiment at least in that the determination unit 113 is included. The configurations and functions of the physical information acquisition unit 111 and the user information acquisition unit 112 are similar to those of the information providing device 100.

In the present example embodiment, the determination unit 113 is a means for determining an optimal behavior pattern for a user's goal using a learning model obtained by machine learning based on life log information. This model is a model obtained using information including the life log information and behavior patterns of the user as the learning data. The determination unit 113 selects an optimal behavior pattern of the user from lifestyle information on calorie intake (IN) in daily meal and calorie consumption (OUT) in basic metabolism and exercise calculated based on the life log information. For example, to reduce about 1000 kcal per week, is required that the value obtained by subtracting OUT from IN described above (IN-OUT) be less than −143 kcal (1000/7 kcal) per day. To achieve this, it may be considered to (1) reduce IN, (2) increase OUT, or (3) reduce IN and increase OUT.

Based on a learning model obtained by machine learning using daily IN and OUT states of a plurality of users as learning data, the determination unit 113 automatically selects which one of the methods (1) to (3) is easy for the user to make the value of IN-OUT negative. This learning model is a model that outputs information on an optimal behavior pattern of the user among (1) to (3) when life log information including meal contents (calorie intake) and exercise contents (calorie consumption) of the user for a predetermined period is input before a determination is made.

Here, a means for determining an optimal behavior pattern among the behavior patterns (1) to (3) described above using the learning model will be described. Using a learning model different for each time j, the learning model determines a behavior pattern to be executed at that time as a behavior pattern for achieving a user's final goal (a goal for achieving a user's ideal situation). The time in this case may be an absolute time or a relative time. In a case where the time is a relative time, the time may be referred to as a stage. The time may refer to a point on the time axis or a predetermined period on the time axis. Hereinafter, j is a natural number. For example, the time j=1 may indicate a first week, the time j=2 may indicate a second week, the time j=t may indicate a t-th week, and the time j=T (T is a natural number larger than t) may indicate a final time, that is, a last week in which whether the final goal has been achieved is known.

For example, a j-th learning model D*ⱼ has a state Xⱼₕ of a user h observed at a time j as an input. Here, the state includes a weight record at each time, an IN record at each time, an OUT record at each time, information on frequency of each meal type at each time, information on frequency of each exercise type at each time, and the like for a user observed from the time 1 to the time j. Then, the j-th learning model D*ⱼ determines a behavior pattern Aⱼₕ of the user h at the time j. The determined behavior pattern Aⱼₕ is a behavior pattern that maximizes the sum (a value obtained by multiplying (IN-OUT) by minus) of effects obtained by the user h from the time j to the final time T.

The determination of the behavior pattern by determination unit 113 is performed positively as time j elapses. For example, if the current time j is t, a state Xₜₕ of the user h observed at the current time t is input to a t-th learning model D*ₜ, thereby obtaining a behavior pattern Aₜₕ of the user h at the current time t. Then, when time t+1 is reached after the time elapses, the determination unit 113 obtains a behavior A₍ₜ₊₁₎ₕ of the user h at the time t+1 by inputting a state X₍ₜ₊₁₎ₕ of the user h observed at the time t+1. In this manner, the determination unit 113 sequentially determines a behavior pattern to be taken as the time elapses. Therefore, a behavior plan is dynamically created.

The decision unit 114 decides a content of an advice based on the optimal behavior pattern of the user. In addition to the method of determining a content of an advice by the decision unit 103, the decision unit 114 decides a content of an advice according to any one of the behavior patterns (1) to (3). For example, when the optimal behavior pattern of the user is (1), the decision unit 114 decides to give an advice mainly on meal content. When the optimal behavior pattern of the user is (2), the decision unit 114 decides to give an advice mainly on exercise content. When the optimal behavior pattern of the user is (3), the decision unit 114 decides to give an advice regarding both meal content and exercise content. Concerning an advice on exercise content in (1) or (2), the decision unit 114 may decide to advise the user to perform the exercise chosen on the screen of FIG. 5. The output unit 115 outputs the advice on meal content and exercise content decided by the decision unit 114.

Furthermore, the determination unit 113 may select an optimal behavior pattern of the user from calorie consumption (OUT) in exercise. In order to achieve this, how much OUT is necessary per day, for example, which one of (A) 100 kcal, (B) 200 kcal, and (C) 300 kcal is necessary as calorie consumption, may be set by exercise type as a behavior pattern. In this case, the determination unit 113 automatically selects which one of the methods (A) to (C) is to be used by the user to achieve the goal based on a learning model obtained by machine learning using information on the daily OUT of the user. The goal mentioned here is, for example, a reduction by 2 kg in one month, the goal being reducing the weight by approximately 2 kg (the goal is not achieved even if the weight is greatly reduced beyond 2 kg). This learning model is a model that outputs information on an optimal behavior pattern of the user among (A) to (C) when life log information including meal contents and exercise contents of the user for a predetermined period is input before a determination is made. Then, the decision unit 114 decides to give an advice on meal contents and exercise contents based on the behavior patterns (A) to (C).

As described above, in the information providing device 110 according to the second example embodiment, the decision unit 114 decides a content of an advice based on an optimal behavior pattern of the user. In this case, it is possible to provide an optimal advice for the goal.

### Third Example Embodiment

Next, a third example embodiment of the present disclosure will be described in detail with reference to the drawings. Hereinafter, description overlapping with what has been described above will be omitted unless the omission obscures the description of the present example embodiment.

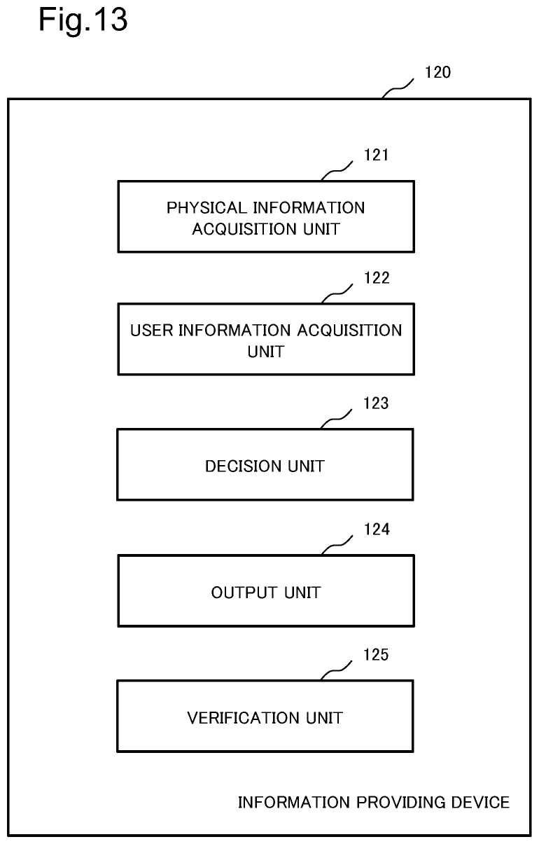

FIG. 13 is a block diagram illustrating an example of a configuration of an information providing device 120 according to the third example embodiment. The function of the information providing device 120 can be implemented not only by hardware similarly to the computer device illustrated in FIG. 2 but also by a computer device based on program control or by software.

As illustrated in FIG. 13, the information providing device 120 includes a physical information acquisition unit 121, a user information acquisition unit 122, a decision unit 123, an output unit 124, and a verification unit 125. The information providing device 120 is different from the information providing device 100 at least in that the verification unit 125 is included. The present example embodiment is similar to the first example embodiment and the first modification of the first example embodiment until the content of the advice decided by the decision unit 123 is output, and the configurations and functions of the physical information acquisition unit 121 and the user information acquisition unit 122 are similar to those of the information providing device 100.

The verification unit 125 is a means for verifying whether the user has executed the output content of the advice. When a predetermined period (several hours or several days) elapses after the output unit 124 outputs the advice, the verification unit 125 acquires information regarding meal content or exercise content for a day or a time zone designated to execute the advice content, for example, based on the life log information. Next, if there is information regarding meal content or exercise content corresponding to the advice content, the verification unit 125 determines that the user has executed the advice content. On the other hand, if there is no information regarding meal content or exercise content corresponding to the advice content, the verification unit 125 determines that the user has not executed the advice content.

When the user has not executed the advised content, the decision unit 123 decides to give an advice different from the advised content. In other words, the decision unit 123 decides a content of a second advice based on whether a first advice already provided to the user has been executed. The content of the first advice is different from the content of the second advice. When an advice regarding meal content has not been executed, the decision unit 123 decides to give an advice to take another food. Furthermore, when an advice regarding exercise content has not been executed, the decision unit 123 decides to give an advice to encourage the user to do another exercise. For example, when the user has not executed an advice given by the decision unit 123 to change melon bread to red bean bread having a smaller calorie intake in order to reduce the calorie intake through the meal, the decision unit 123 decides to give an advice to change the melon bread to a dumpling having a smaller calorie intake.

When the user has not executed an advice on meal content even though the advice on meal content has been continuously given to the user, the decision unit 123 may decide to give an advice on exercise content. Conversely, when the user has not executed an advice on exercise content even though the advice on exercise content has been continuously given to the user, the decision unit 123 may decide to give an advice on meal content. The output unit 124 outputs the decided content of the advice.

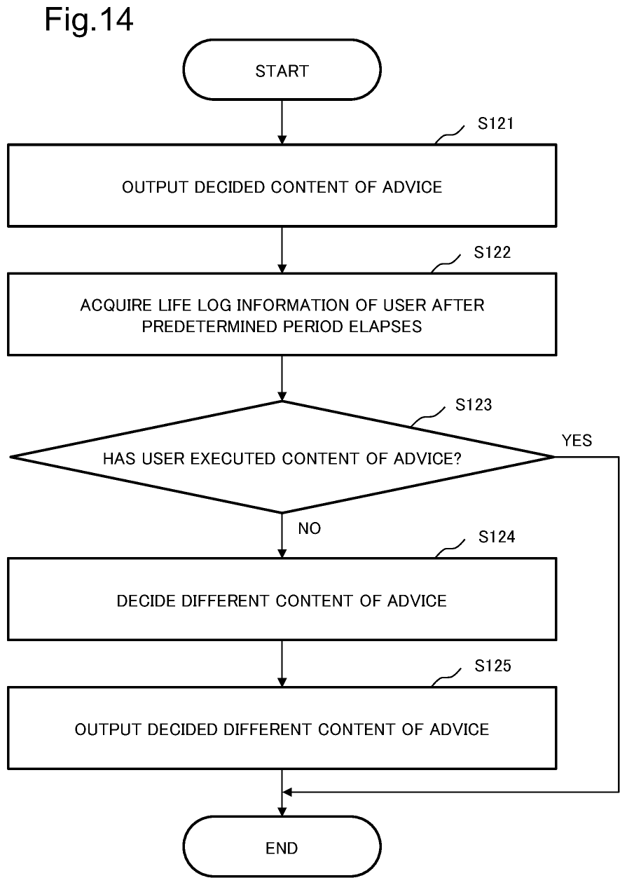

FIG. 14 is a flowchart illustrating an outline of an operation of the information providing device 120 according to the third example embodiment. Note that the process according to this flowchart may be executed based on the program control by the processor described above. This flowchart is performed, for example, after the processing of S108 in FIG. 8. Furthermore, the processing in this flowchart is performed, for example, at a timing of outputting a short-term goal (for example, on a weekly basis).

As illustrated in FIG. 14, first, the output unit 124 outputs an advice content decided by the decision unit 123 (step S121). Next, when a predetermined period elapses after the advice is output, the verification unit 125 acquires life log information of the user (step S122). Next, the verification unit 125 verifies whether the user has executed the output content of the advice (step S123). When the user has not executed the advised content (S123; NO), the verification unit 125 outputs the information to the decision unit 123, and the decision unit 123 decides to give an advice different from the advised content (step S124). Next, the output unit 124 outputs the decided different content of the advice (step S125). On the other hand, when the user has executed the content of the advice (S123; YES), the verification unit 125 ends the process.

As described above, in the information providing device 120 according to the third example embodiment, when the user has not executed a content of an advice, the decision unit 123 gives an advice different from the advised content. In this case, the possibility that the user executes the content of the advice can be increased.

While the present disclosure has been particularly shown and described with reference to exemplary embodiments thereof, the present disclosure is not limited to these embodiments. It will be understood by those of ordinary skill in the art that various changes in form and details may be applied to the example embodiments without departing from the spirit and scope of the present disclosure as defined by the claims. The present disclosure may include example embodiments in which the matters described in the present specification are appropriately combined or replaced if necessary. For example, the matters described using a specific example embodiment can be applied to another example embodiment as long as no contradiction occurs. For example, although a plurality of operations are described in order in the form of a flowchart, the order in which the operations are described does not limit an order in which the plurality of operations are executed. Therefore, when each example embodiment is carried out, the order in which the plurality of operations are executed can be changed within a range that does not interfere with the content.

### REFERENCE SIGNS LIST

100, 110, 120 Information providing device
101, 111, 121 Physical information acquisition unit
102, 112, 122 User information acquisition unit
103, 114, 123 Decision unit
104, 115, 124 Output unit
113 Determination unit
125 Verification unit
500 Computer device
501 CPU
502 ROM
503 RAM
504 Program
505 Storage device
506 Recording medium
507 Drive device
508 Communication interface
509 Input/output interface
510 Bus

What is claimed is:

1. An information providing device comprising:
a memory storing instructions; and
at least one processor configured to execute the instructions to:
acquire physical information including an attribute, a physical condition, and a goal with respect to the physical condition of a user;
acquire information regarding the user including schedule information of the user;
decide a content of an advice for the goal and a timing of executing the content of the advice based on the physical information and the schedule information; and
output the content of the advice and the timing.

2. The information providing device according to claim 1, wherein the user wherein the at least one processor is further configured to execute the instructions to:
acquire life log information of the user, and
acquire the schedule information based on the life log information.

3. The information providing device according to claim 1, wherein the at least one processor is further configured to execute the instructions to:
acquire current behavior information of the user, and
when a behavior for which an advice is providable is detected, decide a content of the advice using the detected behavior.

4. The information providing device according to claim 3, wherein the at least one processor is further configured to execute the instructions to:
acquire life log information of the user, and
decide the content of the advice based on the behavior information and the life log information.

5. The information providing device according to claim 4, wherein the at least one processor is further configured to execute the instructions to:
analyze a lifestyle or preference of the user based on the life log information, and
decide the content of the advice based on the behavior information and lifestyle information or preference information obtained as a result of the analysis.

6. The information providing device according to claim 1, wherein the at least one processor is further configured to execute the instructions to:
acquire environment information regarding a current environment around the user, and
decide the content of the advice based on the environment information.

7. The information providing device according to claim 1, wherein the at least one processor is further configured to execute the instructions to:
acquire life log information of the user,
determine an optimal behavior pattern for the goal by using a model obtained by machine learning based on the life log information, and
decide the content of the advice based on the optimal behavior pattern of the user.

8. The information providing device according to claim 1, wherein the at least one processor is further configured to execute the instructions to:
verify whether the user has executed the output content of the advice, and
wherein when the user has not executed the advised content, give an advice different from the advised content.

9. The information providing device according to claim 1, wherein the at least one processor is further configured to execute the instructions to:
output the content of the advice and the timing for supporting decision making of the user.

10. An information providing method performed by a computer, the information providing method comprising:
acquiring physical information including an attribute, a physical condition, and a goal with respect to the physical condition of a user;
acquiring information regarding the user including schedule information of the user;
deciding a content of an advice for the goal and a timing of executing the content of the advice based on the physical information and the schedule information; and
outputting the content of the advice and the timing.

11. A non-transitory computer-readable recording medium that records a program for causing a computer to execute:
acquiring physical information including an attribute, a physical condition, and a goal with respect to the physical condition of a user;
acquiring information regarding the user including schedule information of the user;
deciding a content of an advice for the goal and a timing of executing the content of the advice based on the physical information and the schedule information; and
outputting the content of the advice and the timing.
